(19) United States
(12) Patent Application Publication
(10) Pub. No.: US 2026/0130836 A1
(43) Pub. Date: May 14, 2026

(54) COMPOSITION, PERSONAL CARE FORMULATIONS, METHOD AND USE FOR TREATING OR PREVENTING DAMAGES TO HAIR

(71) Applicant: Croda International Plc, Goole, East Yorkshire (GB)

(72) Inventors: Patrick James COLVER, Goole, East Yorkshire (GB); Elizabeth Claire RYDER, Goole, East Yorkshire (GB)

(73) Assignee: Croda International Plc, Goole, East Yorkshire (GB)

(21) Appl. No.: 18/868,071

(22) PCT Filed: Jun. 21, 2023
(86) PCT No.: PCT/EP2023/066795

§ 371 Date: Nov. 21, 2024

(30) Foreign Application Priority Data
Jun. 28, 2022 (GB) 2209435.3

(51) Int. Cl. A61K 8/64; A61K 8/362; A61Q 5/00; A61Q 5/02; A61Q 5/12; A61Q 19/00

(52) U.S. Cl. CPC A61K 8/645; A61K 8/362; A61Q 5/002; A61Q 5/02; A61Q 5/12; A61Q 19/00; A61K 2800/34; A61K 2800/524; A61K 2800/95

(57) ABSTRACT

The present invention provides a composition comprising the reaction product of a) itaconic anhydride; and b) an amino compound comprising at least one free amino group, wherein the amino compound is selected from the group consisting of amino acids, peptides and hydrolysed proteins; wherein the composition comprises itaconic modified amino compound, wherein the itaconic modified amino compound is the reaction product of a) and b), and wherein the amount of itaconic anhydride used in the reaction is calculated to react with 10 mol % to 100 mol % of the free amino groups of 0 the amino compound. The invention further provides a personal care formulation comprising the composition, a hair care formulation, a skin care formulation, a method of treating hair or skin and a use of the composition.

### FIELD OF INVENTION

The present invention relates to a composition, personal care formulations comprising the composition such as shampoos or hair conditioners, a method of treating hair or skin with the composition and use of the composition to provide a benefit to hair or skin.

### BACKGROUND

The need for personal care formulations, in particular hair care or skin care products is well-known. Such products may be used for cleaning or conditioning human skin or human hair or addressing various problems, such as those encountered with human hair following treatments such as bleaching or colouring or by exposure to negative environmental factors, such as heat and pollution. Some hair care formulations are also directed at improving qualities of the hair such as strength, shine or ease of brushing/combing.

Silicon-containing compositions may have an unfavourable environmental profile or be otherwise undesirable to use in personal care formulations.

There is a continual need for the development of improved personal care formulations, in particular hair care or skin care formulations, and suitable ingredients for such formulations.

### SUMMARY OF THE INVENTION

The present invention is based in part on the surprising recognition that the reaction product of itaconic anhydride and amino acid containing compounds, particularly hydrolysed proteins, can produce a composition according to the invention which is beneficial in personal care formulations, in particular for the caring and/or conditioning of hair and/or skin. For example, the composition according to the invention may be used as a hair benefit agent in a hair conditioner or shampoo. The benefit provided by the composition of the invention may be selected from hair strengthening, cleaning, conditioning, detangling, repair, shine, vibrancy, softness, anti-frizz, cuticle smoothing, elasticity, humidity control, curl retention, heat protection, improved movement, volume and styling.

Thus viewed from a first aspect, the present invention provides a composition comprising the reaction product of:
a) itaconic anhydride; and
b) an amino compound comprising at least one free amino group, wherein the amino compound is selected from the group consisting of amino acids, peptides and hydrolysed proteins;
wherein the composition comprises itaconic modified amino compound, wherein the itaconic modified amino compound is the reaction product of a) and b), and wherein the amount of itaconic anhydride used in the reaction is calculated to react with 10 mol % to 100 mol % of the free amino groups of the amino compound, preferably calculated by the Formol titre as described herein.

The composition of the invention may be advantageous by not comprising any reactants obtained from a petrochemical source. The itaconic anhydride may be obtained from a renewable source. The itaconic anhydride may be obtained from citric acid. The itaconic anhydride may be obtained from a biological source, preferably from a microbiological source.

Viewed from a second aspect, the present invention provides a personal care formulation comprising a composition of the first aspect.

Viewed from a third aspect, the present invention provides a personal care formulation comprising the reaction product of itaconic anhydride and hydrolysed protein, wherein the personal care formulation comprises from 0.1 to 10 wt % of the reaction product of itaconic anhydride and hydrolysed protein, on the basis of the total weight of the formulation, and wherein the personal care formulation is suitable for topical application.

Viewed from a fourth aspect, the present invention provides a hair care formulation comprising a composition according to the first aspect and a further hair care ingredient.

Viewed from a fifth aspect, the present invention provides a skin care formulation comprising a composition according to the first aspect and a further skin care ingredient.

Viewed from a sixth aspect, the present invention provides a method of treating hair or skin comprising the step of applying a composition according to the first aspect to the hair or skin.

Viewed from a seventh aspect, the present invention provides a method of treating hair, comprising the step of applying a composition according to the first aspect to the hair, wherein the composition provides a benefit to the hair and wherein the benefit is selected from hair strengthening, cleaning, conditioning, detangling, repair, shine, vibrancy, softness, anti-frizz, cuticle smoothing, elasticity, humidity control, curl retention, heat protection, improved movement, volume and styling.

Viewed from an eighth aspect, the present invention provides the use of a composition according to the first aspect of the invention to treat hair or skin to provide a benefit to the hair or skin.

Viewed from a ninth aspect, the present invention provides the use of a composition according to the first aspect to treat hair to provide a benefit to the hair selected from hair strengthening, cleaning, conditioning, detangling, repair, shine, vibrancy, softness, anti-frizz, cuticle smoothing, elasticity, humidity control, curl retention, heat protection, improved movement, volume and styling.

All of the features described herein may be combined with any of the above aspects, in any combination.

### DETAILED DESCRIPTION OF THE INVENTION

It will be understood that any upper or lower quantity or range limit used herein may be independently combined.

It will be understood that, when describing the number of carbon atoms in a substituent group (e.g. ‘C1 to C6’), the number refers to the total number of carbon atoms present in the substituent group, including any present in any branched groups. Additionally, when describing the number of carbon atoms in, for example fatty acids, this refers to the total number of carbon atoms including the one at the carboxylic acid, and any present in any branch groups.

The term ‘residue’ as used herein is the part of a reactant molecule which remains in the reaction product compound after a reaction has occurred.

Many of the chemicals which may be used to produce the composition of the present invention are obtained from natural sources. Such chemicals typically include a mixture of chemical species due to their natural origin. Due to the presence of such mixtures, various parameters defined herein can be an average value and may be non-integral.

The term ‘hydrolysed protein’ is used herein to mean proteins which have been subject to hydrolysis. The hydrolysed protein may comprise protein fragments, polypeptides, peptides, amino acids and/or peptones. The hydrolysed protein may be produced by acid hydrolysis, alkali hydrolysis, and/or enzyme hydrolysis of proteins, preferably naturally occurring proteins or proteins from renewable sources. Enzyme hydrolysis is preferred. Without being bound by theory, an advantage of enzyme hydrolysis when compared with acid or alkali hydrolysis is that the enzyme hydrolysis is more selective in the sites on the protein which are hydrolysed, thus producing an improved amino compound for use in making the composition of the invention when compared with acid or alkali hydrolysis. In general, acid hydrolysis may produce the smallest fragments by weight average molecular weight, alkali hydrolysis may produce the largest fragments, while enzyme hydrolysis may produce fragments of intermediate size between acid and alkali hydrolysis. The size of a fragment in the hydrolysed protein is proportional to the number of amino acid residues in the fragment since the fragments come from the long amino acid chains which make up the unhydrolysed protein. Acid hydrolysis may be disadvantageous due to high temperature and/or pressure requirements. Alkali hydrolysis may be disadvantageous due to the need to use irritant or hazardous chemicals.

The amino compound used in making the composition of the invention may be a chemically unmodified hydrolysed protein. The term ‘chemically unmodified hydrolysed protein’ means a protein that has not been further chemically modified (or reacted) other than by hydrolysis.

The amino compound used in making the composition of the invention may be a partially hydrolysed protein. The term ‘partially hydrolysed protein’ means a protein that has not been hydrolysed completely i.e. not been hydrolysed to the extent that only individual amino acids remain in the amino compound.

The term ‘personal care formulation’ when used herein means a consumer product for the treatment, cleaning, caring or conditioning of the person. The foregoing shall include, but not be limited to, chemicals, compositions, products, or combinations thereof relating to or having use or application in the treatment, cleaning, cleansing, caring or conditioning of the person (including in particular the skin, hair and oral cavity), such as personal wash, hair care and styling, skin care formulations, cosmetics, hygiene products, oral care, and deodorants.

### Composition of the Invention

The composition of the invention comprises the reaction product of:
a) itaconic anhydride; and
b) an amino compound comprising at least one free amino group, wherein the compound is selected from the group consisting of amino acids, peptides and hydrolysed proteins;
wherein the composition comprises itaconic modified amino compound, wherein the itaconic modified amino compound is the reaction product of a) and b), and wherein the amount of itaconic anhydride used is calculated to react with 10 mol % to 100 mol % of the free amino groups of the amino compound. Preferably the amount of itaconic anhydride required is calculated by the Formol titre as described herein.

Preferably the composition further comprises water. The composition may comprise at least 10 wt % water, preferably at least 20 wt % water, more preferably at least 30 wt % water, particularly at least 40 wt % water, desirably at least 50 wt % water, especially at least 60 wt % water, on the basis of the total weight of the composition. The composition may comprise at most 90 wt % water, preferably at most 80 wt % water on the basis of the total weight of the composition.

The composition may further comprise itaconic acid. The itaconic acid may be formed by a minor side-reaction of the itaconic anhydride with water. This side reaction may reduce the amount of itaconic anhydride which actually reacts with the free amino groups of the amino compound by a minor amount, when compared with the theoretical amount calculated by the Formol titre as described herein. Therefore it may be preferable to minimise the side-reaction of itaconic anhydride with water by selecting suitable reaction conditions. Preferably the amount of itaconic anhydride added to react with the amino compound is the theoretical amount calculated to react with a specified mol % of the amino groups in the amino compound. Preferably the theoretical amount is calculated by the Formol titre.

The itaconic anhydride may be obtained from a renewable source. Preferably the itaconic anhydride is not obtained from a petrochemical source. The itaconic anhydride may be obtained from citric acid. The itaconic anhydride may be obtained from a biological source, preferably from a microbiological source. Preferably the itaconic anhydride is substantially pure itaconic anhydride, for example at least 85 wt % itaconic anhydride, preferably at least 90 wt % itaconic anhydride, more preferably at least 95 wt % itaconic anhydride.

Preferably the amino compound is obtained from a renewable source. Preferably the amino compound is not obtained from an animal protein source. This is advantageous since animal sources can be undesirable for consumers. Preferably the composition comprises no animal-derived components. Preferably the composition comprises no petrochemical-derived components. Preferably the carbon-containing parts of the composition are at least 80% biobased according to ASTM D6866 on the basis of the total weight of the carbon-containing parts of the composition, more preferably at least 90%, particularly at least 95%, desirably at least 99%, especially 100% biobased. Preferably the composition is suitable for vegan consumers.

Preferably the amino compound comprises hydrolysed protein, more preferably consists essentially of hydrolysed protein, more preferably is hydrolysed protein. The hydrolysed protein may be produced by acid, alkali or enzyme hydrolysis, preferably acid or enzyme hydrolysis. Enzyme hydrolysis is particularly preferred. One or more enzymes may be used. The enzyme is preferably not animal derived. Preferably the enzyme is from a micro-organism source. The enzyme(s) may comprise a carbohydrase and/or a protease. Preferably the enzyme comprises a protease. The hydrolysis may be performed to the extent required to achieve the desired weight average molecular weight of the hydrolysed protein. The extent of hydrolysis may be varied by varying the temperature, acid/alkali/enzyme used, and time taken. The resulting hydrolysed protein may be filtered and/or treated to remove undesired material. For example the hydrolysed protein may be treated to remove any chloride ions present if acid hydrolysis is used.

The hydrolysed protein may be obtained from a natural source. The hydrolysed protein may be obtained from a renewable source. Preferably the hydrolysed protein is obtained from a vegetable source. The vegetable source may be selected from potato, wheat, cottonseed, pea, chickpea and soya, preferably selected from potato, wheat, pea and chickpea more preferably selected from potato and chickpea. Preferably the amino compound is a hydrolysed vegetable protein, preferably a hydrolysed potato protein. The composition may not comprise hydrolysed wheat protein or protein obtained from a wheat source. This may be advantageous since wheat protein may be undesirable for certain consumers.

Preferably the amino compound comprises oligomers of amino acids, which may also be known as oligopeptides. These oligomers may be hydrolysed protein fragments. The amino compound may comprise oligomers of 2 to 20 amino acids (also known as residues) in average size, preferably of 2 to 15 amino acids, more preferably of 2 to 10 amino acids. Preferably the average size is a weight average size. The amino acids in the oligomers may be the same or different amino acids. The amino acids in the oligomers preferably comprise at least one glutamic acid residue or histidine residue, more preferably comprise at least one glutamic acid residue. The amino compound may not be an individual amino acid. Preferably the amino compound does not consist of individual amino acids. Alternatively, the amino compound may comprise glutamic acid or histidine as individual amino acids, preferably glutamic acid.

The weight average molecular weight (Mw) of the amino compound may be at least 100 Daltons (Da), preferably at least 200 Da, preferably at least 300 Da. The weight average molecular weight of the amino compound may be at most 20,000 Da, preferably at most 10,000 Da, preferably at most 8,000 Da, preferably at most 5,000 Da, preferably at most 3000 Da, preferably at most 1500 Da, preferably at most 1000 Da. The molecular weight may be measured by size exclusion chromatography, preferably size-exclusion HPLC (SE-HPLC) as described in the Test Methods below. Preferably the amino compound has a weight average molecular weight in the range from 100 Da to 5000 Da, preferably 100 Da to 3000 Da, preferably 100 Da to 1500 Da.

Preferably, the composition of the invention is substantially free from quaternary ammonium compounds. By the use of the term “substantially free from”, it is meant that the composition comprises preferably less than 10% by weight, more preferably less than 5% by weight, yet more preferably less than 2% by weight and most preferably, less than 1% by weight based on the total weight of the composition. Preferably, the composition does not comprise a quaternary ammonium compound.

Preferably, the composition of the invention is substantially free from silicone compounds. By the use of the term “substantially free from”, it is meant that the composition comprises preferably less than 10% by weight, more preferably less than 5% by weight, yet more preferably less than 2% by weight and most preferably, less than 1% by weight based on the total weight of the composition. Preferably, the composition comprises no silicone compounds. Preferably the composition does not comprise an organosilane, organosilicon or silicone compound.

The composition may further comprise one or more preservatives. The composition may comprise at least 0.1 wt % preservative, preferably at least 0.2 wt %, more preferably at least 0.4 wt %, particularly at least 0.6 wt % on the basis of the total weight of the composition. The composition may comprise at most 10 wt % preservative, preferably at most 5 wt %, more preferably at most 3 wt %, particularly at most 2 wt % on the basis of the total weight of the composition. The preservative may comprise phenoxyethanol, a benzoate or a sorbate, more preferably the preservative is selected from benzoates and sorbates, more preferably the preservative comprises sodium benzoate and/or potassium sorbate. Alternatively, the composition may not comprise a preservative.

The composition may be a liquid at room temperature (25° C.). The composition may be a solution or dispersion, preferably a solution, of the itaconic modified amino compound in a solvent, preferably the solvent comprises water.

Alternatively the composition may not comprise water or may comprise residual water after drying to a solid or powder form. Spray drying may be used to dry the composition. The composition may be a solid or powder.

### Ratios of Reactants

The reactants for the composition of the invention comprise:
a) itaconic anhydride; and
b) an amino compound comprising at least one free amino group, wherein the compound is selected from the group consisting of amino acids, peptides and hydrolysed proteins.

The composition comprises itaconic modified amino compound, wherein the itaconic modified amino compound is the reaction product of a) and b), and wherein the amount of itaconic anhydride used is calculated to react with 10 mol % to 100 mol % of the free amino groups of the amino compound. Preferably the calculation is by Formol titre as described herein.

The ratio of reactants may surprisingly influence the properties of the composition of the invention. At least 10 mol %, more preferably at least 15 mol %, more preferably at least 20 mol %, more preferably at least 40 mol %, more preferably at least 60 mol %, more preferably at least 70 mol % of the free amino groups in the amino compound may be reacted with the itaconic anhydride in the itaconic modified amino compound. At most 99 mol %, preferably at most 98 mol %, more preferably at most 95 mol % of the free amino groups in the amino compound may be reacted with the itaconic anhydride in the itaconic modified amino compound. Preferably the amount of itaconic anhydride used is calculated to react with 40 mol % to 100 mol % of the free amino groups in the amino compound. More preferably the amount of itaconic anhydride used is calculated to react with 60 mol % to 99 mol % of the free amino groups in the amino compound. Preferably the calculation is by Formol titre as described herein.

### Personal Care Formulations

Preferably the composition of the invention is suitable for use in personal care formulations. Viewed from a second aspect, the present invention provides a personal care formulation comprising the composition of the invention. Preferably the personal care formulation is for topical application to skin or hair.

The personal care formulation may be selected from moisturizers, body butters, gel creams, high perfume containing products, perfume creams, hair conditioners, hair relaxer formulations, hair shampoos, hair styling products, leave-on hair products, water-free products, anti-perspirant and deodorant products, cleansers, 2-in-1 foaming emulsions, emulsifier free products, mild formulations, scrub formulations e.g. containing solid beads, silicone in water formulations, pigment containing products, sprayable emulsions such as hair detanglers, colour cosmetics, shower products, make-up remover, eye make-up remover, and wipes. Preferably, the personal care formulation is selected from hair conditioners, hair relaxer formulations, hair shampoos, hair styling products, leave-on hair products and sprayable emulsions such as hair detanglers.

Preferably, the composition of the invention is present in the personal care formulation at a concentration of at least 0.01% w/w, preferably at least 0.1% w/w, more preferably at least 0.5% w/w and most preferably at least 0.8% w/w based on the total weight of the formulation. Preferably, the composition of the invention is present in the personal care formulation at a concentration of up to 20% w/w, preferably up to 15% w/w, more preferably up to 10% w/w and most preferably up to 8% w/w based on the total weight of the formulation.

Preferably the personal care formulation comprises the reaction product of itaconic anhydride and hydrolysed protein, wherein the personal care formulation comprises from 0.01 to 10 wt %, preferably 0.1 to 10 wt %, of the reaction product of itaconic anhydride and hydrolysed protein, on the basis of the total weight of the formulation, and wherein the personal care formulation is suitable for topical application.

The invention may provide a personal care formulation comprising the composition of the invention and a personal care ingredient. The personal care ingredient may be selected from a cleaning agent, hair conditioning agent, hair styling agent, anti-dandruff agent, hair growth promoter, perfume, pigment, moisturizer, film former, hair color, make-up agent, thickening agent, emulsifier, humectant, emollient, antiseptic agent, deodorant active, dermatologically acceptable carrier, surfactant, abrasive, absorbent, fragrance, colorant, essential oil, astringent, anti-acne agent, anti-caking agent, anti-foaming agent, anti-oxidant, binder, enzyme, enzyme inhibitor, enzyme activator, coenzyme, botanical extract, ceramide, buffering agent, bulking agent, chelating agent, cosmetic biocide, external analgesic, substantivity increasing agent, opacifying agent, pH adjuster, reducing agent, sequestrant, skin bleaching and/or lightening agent, skin conditioning agent, skin soothing and/or healing agent, skin treating agent, vitamin or preservative.

Preferably the personal care ingredient is selected from a cleaning agent, hair conditioning agent, skin conditioning agent, hair styling agent, antidandruff agent, hair growth promoter, perfume, pigment, moisturizer, film former, humectant, alpha-hydroxy acid, hair colour, make-up agent, thickening agent, antiseptic agent, deodorant, surfactant.

Many other personal care ingredients may be used in the personal care formulations according to the present invention. These ingredients may be oil soluble, water soluble or non-soluble. Examples of such ingredients include:
(i) preservatives, preferably those approved for cosmetic use, particularly as listed in Annex 5 of the European Union cosmetics regulations. The preservative preferably comprises sodium benzoate, potassium sorbate or phenoxyethanol. The preservative may comprise parabens (alkyl esters of 4-hydroxybenzoic acid), substituted ureas or hydantoin derivatives, e.g. those sold commercially under the trade names Germaben II, Nipaguard BPX and Nipaguard DMDMH. The preservative may be used at a concentration in the range from 0.5 wt. % to 2 wt. % based on the total weight of the formulation;
(ii) perfumes, when used typically at a concentration in the range from 0.1 wt. % to 10 wt. % more usually up to about 5 wt. % and particularly up to about 2 wt. %, based on the total weight of the formulation;
(iii) humectants or solvents such as alcohols, polyols such as glycerol and polyethylene glycols, when used typically at a concentration in the range from 1 wt. % to 10 wt. % based on the total weight of the formulation;
(iv) alpha hydroxy acids such as glycolic, citric, lactic, malic, tartaric acids and their esters; self-tanning agents such as dihydroxyacetone;
(v) vitamins and their precursors including: (a) Vitamin A, e.g. as retinyl palmitate and other tretinoin precursor molecules, (b) Vitamin B, e.g. as panthenol and its derivatives, (c) Vitamin C, e.g. as ascorbic acid and its derivatives, (d) Vitamin E, e.g. as tocopheryl acetate, (e) Vitamin F, e.g. as polyunsaturated fatty acid esters such as gamma-linolenic acid esters;
(vi) skin care agents such as ceramides either as natural materials or functional mimics of natural ceramides;
(vii) natural phospholipids, e.g. lecithin;
(viii) vesicle-containing formulations;
(ix) botanical extracts with beneficial skin care properties;
(x) skin whiteners such as kojic acid, arbutin and similar materials;
(xi) skin repair compounds actives such as Allantoin and similar series;
(xii) caffeine and similar compounds;
(xiii) cooling additives such as menthol or camphor;
(xiv) insect repellents such as N, N-diethyl-3-methylbenzamide (DEET) and citrus or eucalyptus oils;
(xv) essential oils; and
(xvi) pigments, including microfine pigments, particularly oxides and silicates, e.g. iron oxide, particularly coated iron oxides, and/or titanium dioxide, and ceramic materials such as boron nitride, or other solid components, such as are used in make-up and cosmetics, to give suspoemulsions, typically used in an amount in the range from 1 wt. % to 15 wt. %, but usually at least 5 wt. %, and particularly about 10 wt. % based on the total weight of the formulation.

The personal care formulations may comprise a perfume or fragrance-imparting material to provide a pleasant scent. In one aspect, a perfume is provided from a natural source, such as but not limited to alfalfa, almond, amber, angelica root, anise, apple, apricot, banana, basil, bay, bay laurel, benzoin, bergamot, bitter orange, black pepper, bois de rose (rosewood), cajeput, cardamom, carrot seed, cedarwood, cinnamon, citronella, citrus, clary sage, clove, cocoa, coconut, coffee, coriander, cranberry, cypress, elemi, eucalyptus globulous, eucalyptus, fennel, frankincense, galbanum, geranium, German chamomile, ginger, grapefruit, helichrysum, hyssop, jasmine, juniper berry, lavender, lemon, lemongrass, lily, linden blossom, mango, marjoram, melissa, mint, myrrh, myrtle, neroli, niaouli, nutmeg, orange, oregano, palm, parsley, patchouli, peach, peppermint, petitgrain, pine, pineapple, raspberry, Roman chamomile, rose, rosemary, sandalwood, spearmint, spruce, strawberry, tea, thyme, vanilla, vetiver, violet, yarrow, ylang, and the like. Preferably, the fragrance is selected from mint or vanilla.

Preferably the personal care formulation is a skin care formulation. The invention may provide a skin care formulation comprising the composition of the invention and a skin care ingredient. Preferably the skin care ingredient is as described herein. Preferably the skin care formulation is for topical application to skin.

Preferably, the composition of the invention is present in the skin care formulation at a concentration of at least 0.01% w/w, preferably at least 0.1% w/w, more preferably at least 0.5% w/w and most preferably at least 0.8% w/w based on the total weight of the formulation. Preferably, the composition of the invention is present in the skin care formulation at a concentration of up to 20% w/w, preferably up to 15% w/w, more preferably up to 10% w/w and most preferably up to 8% w/w based on the total weight of the formulation.

Preferably the personal care formulation is a hair care formulation. The invention may provide a hair care formulation comprising the composition of the invention and a hair care ingredient. Preferably the hair care formulation is for topical application to hair.

The hair care ingredient may be selected from shine enhancers, moisturisers, herbal additives, hair strengtheners, vitamin additives, colorants, hair thickening agents; setting and styling agents; ultraviolet absorbers; silicone oils; essential oils and fragrances; thickening or viscosity-enhancing agents; detergents; stabilising agents; emollients; chelating agents; sequestering agents; preservatives; disinfectants; anti-oxidants/radical scavengers; antistatic agents; conditioning agents; detangling ingredients; emulsifying or dispersing agents.

Preferably the hair care formulation is a hair cleansing, conditioning, de-tangling, colour-protecting or styling formulation, particularly preferably a hair cleansing or conditioning formulation.

Preferably, the composition of the invention is present in the hair care formulation at a concentration of at least 0.01% w/w, preferably at least 0.1% w/w, more preferably at least 0.5% w/w and most preferably at least 0.8% w/w based on the total weight of the formulation. Preferably, the composition of the invention is present in the hair care formulation at a concentration of up to 20% w/w, preferably up to 15% w/w, more preferably up to 10% w/w and most preferably up to 8% w/w based on the total weight of the formulation.

The formulation may be in the form of an aqueous “leave on” or an aqueous “rinse off” end-use product. For such formulations, a dilute solution may be used. Preferably, a buffered solution is used, in which the pH of the solution is adjusted to mildly acidic, with a pH in the range of from 4 to 6. In the case of rinse-off formulations, instructions are provided to wash off the diluted formulation after application. Depending on the level of treatment required, such instructions may also require the product to remain on the hair for some time, such as from 1 to 30 minutes. For leave-on formulations, the washing off step is omitted.

Where the formulation is a hair shampoo or conditioner which functions to make the hair straighter, the shampoo or conditioner may be in the form of a dispersion, emulsion or solution. One preferred system is one that forms liquid crystals. The liquid crystals are preferably lyotropic liquid crystals (i.e. both concentration and temperature dependent), more preferably lamellar phase liquid crystals, and particularly L-alpha phase (neat) liquid crystals.

The hair care formulation may contain many different types of hair care ingredients such as;
(i) cationic hair conditioning agents, e.g. ethoxylated phosphate fatty quats, such as those sold by Croda as Arlasilk™; fatty amido amines, such as those sold by Croda as Incromine™; fatty quats, such as those sold by Croda as Incroquat™, Crodazosoft™, Rejuvasoft™ or VibraRiche™ typically used at a concentration in the range from 1 wt. % to 5 wt. % based on the total weight of the formulation. These are typically combined with polymeric hair conditioning cationic materials such as quaternised cellulose sold by Croda as Crodacel™ and MiruStyle™, quaternised proteins, such as those sold by Croda, as Croquat™, Crosilkquat™, Kerestore™ and Hydrotriticum™.
(ii) fatty alcohols, e.g. stearyl, cetearyl, cetyl, oleyl alcohols, used typically at a concentration range of 2 wt. % to 5 wt. % based on the total weight of the formulation.
(iii) humectants or solvents, e.g. alcohols and polyols such as glycerol and polyethylene glycols, when used typically at a concentration in the range from 1 wt. % to 10 wt. % based on the total weight of the formulation;
(iv) reconstructors, e.g. hydrolysed proteins such as wheat protein, which function to penetrate the hair and strengthen the hair structure through polymer crosslinking;
(v) glossing or detangling materials which bind to the hair and reflect light, e.g. silicones such as dimethicone, phenyltrimethicone, dimethiconol and/or trimethylsilylamodimethicone, usually at a concentration in the range from 0.2 wt. % to 10 wt. % based on the total weight of the formulation;
(vi) acidity regulators, e.g. citric acid, lactic acid, which generally maintain the pH of the conditioner at about 4 to 6;
(vii) thermal protectors, usually heat-absorbing polymers, which shield the hair against excessive heat, e.g. caused by blow-drying or curling irons or hot rollers such as for instance those sold by Croda as Mirustyle™ MFP (quaternised starch); and
(viii) UV protection agents, to protect hair or formulation components from degradation by UV light, such as those sold by Croda as Crodasorb™ UV-HPP.

### Shampoo Formulations

A preferred personal care formulation according to the invention is a shampoo comprising 0.1 to 20 wt % of the composition of the invention on the basis of the total weight of the formulation and further comprising:
a) at least one surfactant selected from:
   i) anionic surfactants in a total amount of 0.1 to 30 wt %, preferably 0.5 to 20 wt %, more preferably 1 to 15 wt %, more preferably 3 to 10 wt %, more preferably 5 to 7 wt %, on the basis of the total weight of the formulation, wherein the at least one anionic surfactant is selected from alkyl sulphates, sulfonates and phosphates, alkyl polyglycol ether sulfates and ether carboxylic acids having 10 to 18 C atoms in the alkyl group and 2 to 6 ethylene oxide groups in the molecule, acyl isethionates having 8 to 24 C atoms in the acyl group, and acylated amino acids and protein fatty acid condensates having 8 to 24 C atoms in the acyl group and from mixtures of these anionic surfactants;
   ii) non-ionic surfactants selected from alkanol amides, sugar-based surfactants and ethoxylated surfactants; and
   iii) zwitterionic surfactants in a total amount of 0.1 to 15 wt %, preferably 0.2 to 10 wt %, more preferably 0.5 to 8 wt %, more preferably 1 to 5 wt %, more preferably 2 to 4 wt %, on the basis of the total weight of the formulation, wherein the zwitterionic surfactant is selected from amphoacetates/amphodiacetates, amine oxides, sultaines and betaines, more preferably the zwitterionic surfactant comprises cocamidopropyl betaine.
b) at least one further hair care ingredient as described herein; and
c) water.

### Hair Conditioner Formulations

A preferred personal care formulation according to the invention is a rinse-off hair-conditioner comprising 0.1 to 20 wt % of the composition of the invention on the basis of the total weight of the formulation and further comprising:
a) at least one surfactant selected from:
   i) cationic surfactants in a total amount of 0.01 to 8 wt %, preferably 0.1 to 6 wt %, more preferably 0.5 to 5 wt %, more preferably 1.0 to 4 wt %, more preferably 1.5 to 3.5 wt %, with respect to the total weight of the formulation, wherein preferably the cationic surfactant is selected from quaternary ammonium compounds having at least one C8-C24 alkyl residue, esterquats and amidoamines having at least one C8-C24 acyl residues,
   ii) non-ionic surfactants selected from alkanol amides, sugar-based surfactants and ethoxylated surfactants;
b) optionally, at least one linear saturated 1-alkanol having 12 to 30 carbon atoms in a total amount of 0.2 to 10 wt %, especially preferably 0.5 to 7 wt %, with respect to the total weight of the formulation, wherein at least one linear saturated 1-alkanol selected from lauryl alcohol, myristyl alcohol, cetyl alcohol, stearyl alcohol, arachidyl alcohol, and behenyl alcohol and from mixtures of these 1-alkanols is included, and
c) optionally at least one oil in a total amount of 0.01 to 10 wt %, preferably 0.1 to 3 wt %, especially preferably 0.5 to 1 wt %, with respect to the total weight of the formulation; and
d) water.

### Method of Treating Hair or Skin

Viewed from another aspect, the present invention provides a method of treating hair or skin comprising the step of applying a composition according to the invention to the hair or skin.

Preferably the composition provides a benefit to the hair or skin and wherein the benefit is selected from cleaning and conditioning.

Preferably the composition comprises any of the features described herein with regard to the composition of the invention.

The method may comprise rinsing the composition from the hair or skin using water. Alternatively the method may comprise leaving the composition on the hair or skin.

Viewed from another aspect, the present invention provides a method of treating hair, comprising the step of applying a composition according to the invention to the hair, wherein the composition provides a benefit to the hair and wherein the benefit is selected from hair strengthening, cleaning, conditioning, detangling, repair, shine, vibrancy, softness, anti-frizz, cuticle smoothing, elasticity, humidity control, curl retention, heat protection, improved movement, volume and styling.

Preferably the composition is part of a hair shampoo or conditioner formulation, preferably comprising any of the features described herein.

Preferably the composition comprises any of the features described herein with regard to the composition of the invention.

### Use of the Composition to Treat Hair or Skin

Viewed from another aspect, the present invention provides the use of a composition according to the invention to treat hair or skin to provide a benefit to the hair or skin selected from cleaning and conditioning.

Viewed from another aspect, the present invention provides the use of a composition according to the invention to treat hair to provide a benefit to the hair selected from hair strengthening, cleaning, conditioning, detangling, repair, shine, vibrancy, softness, anti-frizz, cuticle smoothing, elasticity, humidity control, curl retention, heat protection, improved movement, volume and styling.

Preferably the composition is part of a hair shampoo or conditioner formulation, preferably comprising any of the features described herein.

Preferably the composition comprises any of the features described herein with regard to the composition of the invention.

### EXAMPLES

The invention is illustrated by the following non-limiting examples. All parts and percentages are given by weight unless otherwise stated.

It will be understood that all tests and physical properties listed have been determined at atmospheric pressure and ambient temperature (i.e. about 23° C.), unless otherwise stated herein, or unless otherwise stated in the referenced test methods and procedures.

### Test Methods

In this specification the following test methods have been used:
(i) Formol titre a titration method to determine the degree of substitution of amino groups described by Cobbett, W. G., Gibbs, J. A. and Leach, A. A. (1964). J. Appl. Chem. (London), 14, 296-302
(ii) pH was measured at room temperature (25° C.) with Fisher Scientific Accumet AE150 PH Meter. Depending on the initial pH reading, the pH of the formulation was adjusted to the range of 4.0-5.0 using 10 wt % sodium hydroxide solution (if the pH is higher) or 10 wt % citric acid solution (if the pH is lower). The pH was measured immediately and 24 hours after making the formulation.
(iii) Actives content (in wt %) in the sample was calculated from measuring the total non-volatile content & ash content. Total non-volatile content was measured for a known weight of sample by oven drying at 105° C. for 17-19 hours to remove the moisture and any other volatile components present. After cooling in a desiccator, the residual weight is used to calculate the total non-volatile content (in wt %) of the sample. For ash content, a known weight of sample is heated carefully, using incremental temperature steps, to a temperature of 575-600° C. in the presence of air and held at temperature for 16-18 hours (or longer if necessary). After cooling in a desiccator, the residual weight is used to calculate the ash content (in wt %) of the sample. The actives content (in wt %) in the sample is then calculated by subtracting wt % ash from wt % total non-volatile content.
(iv) Weight average molecular weight was determined by Size-Exclusion High-Performance Liquid Chromatography (SE-HPLC). The HPLC apparatus and settings used are given in Table 1.

TABLE 1
HPLC apparatus and settings

| Spectrometer | Agilent 1200 series HPLC | |
|---|---|---|
| Detector | UV @ 220 nm | |
| Columns | Superdex 30 Increase | |
| Solvent | 0.1% TFA, 0.1M Na₂SO₄, 22% MeCN | |
| Concentration of | 0.02 | g/ml |
| test substance | | |
| Column temperature | 25° | C. |
| Flow rate | 0.8 | ml/min |
| Injection Volume | 10 | μL |
| Analysis time | 45 | minutes |

| | |
|---|---|
| Method control | Agilent OpenLab software |
| and integration | |
| Method Type | Peak area integration |
| Calibration | The following standards (8 points) are used to |
| | calibrate the curve using polynomial 3: 12,384 |
| | (Da) Cytochrome C, 1,423 Bacitracin, 433 Arg- |
| | Gly-Asp-Ser, 146 Glutamine, 3496 Insulin |
| | Chain B, 1355 Vitamin B12, 189 (Gly)3, 75 |
| | Glycine |

(v) Single hair fiber tensile test data was measured using a Tensile Tester (MTT690), equipped with Automatic Loading System (model ALS1500) and Fiber Dimensional Analysis System (model FDAS770) from Diastron Ltd. (Andover, Hants, UK) in a controlled environment chamber. 50 random single fibers from each hair tress, treated with each sample, were first prepared for testing with AAS 1600 (Auto-Assembly System) from Diastron Ltd. Each hair fiber sample had specification of 30 mm in length. The prepared hair fiber samples were then loaded onto the rotary sample tray and pre-equilibrated at 50% relative humidity (RH) and room temperature (˜25° C.) for at least 1 hour prior to performing the experiment. After pre-equilibration, each fiber sample was moved from the sample tray to FDAS770 using ALS1500. The FDAS770 employed a laser micrometer to measure dimensions of the fiber sample (cross-sectional area in square microns) by scanning 5 slices along the length of the fiber. The fiber sample was then moved back to the sample tray, where a pneumatically operated rod holds one end of the fiber sample taut while a pneumatically operated jaw equipped with 2 kg (20 N) load cell grabs onto the other end of the fiber sample, and slowly stretched the hair fiber sample at 20 mm/min extension rate until the fiber reached break point. At break point, each fiber sample generated a stress-strain curve. Data for Break Stress (gmf/sq micron) was generated, defined as the stress at which the hair fibre will break.

### Example 1

The amino compound which is used to make the composition of the invention may be made by any of Methods A to D below.

### Method A

Potato protein concentrate was added to a hydrochloric acid solution and mixed until well dispersed. The resulting dispersion was heated to reflux conditions. The dispersion was heated until the weight average molecular weight (measured by SE-HPLC as described in the test methods) of the protein stopped reducing, usually in the range of 200-500 Da. Once the hydrolysis was complete the pH of the solution was increase by the addition of sodium hydroxide. The protein hydrolysate was purified by filtration and treatment with activated carbon. Once purified, more sodium hydroxide was added to make the solution basic, and the solution was concentrated to the desired active content (in the range 20 to 30 wt % actives content) by evaporation or ultra-filtration with ultra-filtration having the benefit of salt removal. The resulting product will be referred to as amino compound A

### Method B

Potato protein concentrate was added to a hydrochloric acid solution and mixed until well dispersed. The resulting dispersion was heated to reflux conditions. The dispersion was heated until the weight average molecular weight of the protein stopped reducing, usually in the range of 200-500 Da. The acidic protein hydrolysate was then recovered by filtration. The pH of the solution was then increased by passing the solution through an ion exchange resin to remove the chloride ions. The protein hydrolysate was purified by treatment with activated carbon. Once purified, more sodium hydroxide was added to make the solution basic, and the solution was concentrated to the desired active content (in the range 20 to 30 wt % actives content). The resulting product will be referred to as amino compound B.

### Method C

Potato protein concentrate was added to water and mixed until well dispersed. The resulting dispersion was heated to the desired temperature before the pH was adjusted using sodium hydroxide solution. Enzymes (carbohydrase and protease) were used to catalyse hydrolysis of the potato protein by stirring at the desired temperature and pH ranges to achieve a typical weight average molecular weight of about 400 to 700 Da. Following hydrolysis, the pH of the hydrolysis mixture was lowered to acidic through addition of hydrochloric acid solution and the enzymes were denatured via heating. The protein hydrolysate was purified by filtration and treatment with activated carbon. The resulting dilute hydrolysed protein solution was then adjusted to a basic pH using sodium hydroxide and then concentrated to the desired actives content (in the range 20 to 30 wt % actives content). The resulting product will be referred to as amino compound C.

### Method D

Potato protein concentrate was added to water and mixed until well dispersed. The resulting dispersion was heated to the desired temperature before the pH was adjusted using sodium hydroxide solution. Enzymes (carbohydrase and protease) were used to catalyse hydrolysis of the potato protein by stirring at the desired temperature and pH ranges to achieve a typical weight average molecular weight of about 700 to 950 Da. Following hydrolysis, the pH of the hydrolysis mixture was lowered to acidic through addition of hydrochloric acid solution and the enzymes were denatured via heating. The protein hydrolysate was purified by filtration and treatment with activated carbon. The resulting dilute hydrolysed protein solution was then adjusted to a basic pH using sodium hydroxide and then concentrated to the desired actives content (in the range 20 to 30 wt % actives content). The resulting product will be referred to as amino compound D.

### Example 2

In a suitable vessel, amino compound A, B, C or D is reacted with itaconic anhydride to produce the composition of the invention according to the following method:
1. Load the amino compound into the vessel.
2. Adjust pH to 9.5-11.0 (preferably 10.0-10.5) with sodium hydroxide. A high pH is preferred to encourage the itaconic anhydride to react with the amino compound instead of with water to avoid or reduce the production of itaconic acid.
3. Add calculated quantity of itaconic anhydride (calculated on the Formol titre, a technique described by Cobbett, W. G., Gibbs, J. A. and Leach, A. A. (1964). J. Appl. Chem. (London), 14, 296-302). This calculation is based on the reaction proceeding to completion, but some itaconic anhydride may react with water to form itaconic acid and prevent the complete reaction of the itaconic anhydride with the amino compound. An example of the calculated quantity of itaconic anhydride required in grams is e.g. 200 (exemplary weight in g of amino compound)×55 (exemplary result from Formol titre)×0.02 (molarity of NaOH used in Formol titre)×112 (molecular weight of itaconic anhydride)×0.75 (75 mol % of free amino groups required to react)/1000 (unit conversion).
4. Maintain temperature at 15-45° C., preferably 20-35° C., and pH at 9.5-11.0, preferably 10.0-10.5, during the itaconic anhydride addition and reaction initiation.
5. Maintain reaction conditions for a further 1-4 hours (preferably 1-2 hours).
6. Acidify to pH 3.5-5.5 (preferably 4.0-5.0) with a mineral acid, such as hydrochloric acid.
7. Preserve by addition of a preservative.
8. Cool and adjust pH to pH 3.5-5.5 (preferably 4.0-5.0).
9. Filter.

The samples in Table 2 below were prepared using the methods from Example 1 and 2, except Sample 1 which was without itaconic modification and so only used Example 1. All Samples were adjusted to between pH 3.5-5.

TABLE 2
Samples prepared

| Sam- | | Preparation method used |
|---|---|---|
| ple | Composition | from Example 1 |
| 1 | Amino Compound B without itaconic | Method B |
| | modification | |
| 2 | 100 mol % Itaconic Modified Amino | Method A with additional |
| | Compound A | ion exchange purification |
| 3 | 100 mol % Itaconic Modified Amino | Method A |
| | Compound A | |
| 4 | 100 mol % Itaconic Modified Amino | Method D |
| | Compound D | |
| 5 | 50 mol % Itaconic Modified Amino | Method A |
| | Compound A | |
| 6 | 75 mol % Itaconic Modified Amino | Method A |
| | Compound A | |
| 7 | 90 mol % Itaconic Modified Amino | Method C |
| | Compound C | |

### Example 3

Hair samples were treated and tensile tested using the following procedure:
a) Bleach European brown, non-damaged hair to the desired damage level using a commercial bleaching system (Clynol Viton Cream Peroxide 12% and Clynol Viton Blue Powder Bleach)
b) Divide a single hair tress into multiple mini-tresses to undergo different treatments and be compared
c) Saturate each mini tress in a solution containing 1% (w/w) active for 30 minutes
d) For a control, saturate a mini-tress in DI water for 30 minutes
e) Use a hair dryer to dry each mini-tress using a low heat, high power setting

Hair tensile test data was measured as described in the Test Method section above taking 50 fibres from each mini tress.

The results were compared to the DI water control. The average results are given in Table 3 below.

TABLE 3
Hair Test Results Leave-On Treatment

| Sample | Composition | Break Stress (gmf/sq micron) |
|---|---|---|
| Control | De-ionised Water | 1.907E−02 |
| 1 | Amino Compound B without | 1.953E−02 |
| | itaconic modification | |
| 3 | 100 mol % Itaconic Modified | *1.976E−02 |
| | Amino Compound A | |

*denotes a significant improvement compared to the control (Student's t test with 95% confidence interval)

Table 3 displays the increase in break stress of hair treated with Amino Compound B. A further improvement is seen with itaconic modified Amino Compound A. Hair treated with itaconic modified Amino Compound A has a significantly higher break stress compared to the control.

### Example 4

Hair samples were treated and tensile tested using the following procedure:
a) Bleach European brown, non-damaged hair to the desired damage level using a commercial bleaching system (Clynol Viton Cream Peroxide 12% and Clynol Viton Blue Powder Bleach)
b) Divide a single hair tress into multiple mini tresses to undergo different treatments and be compared
c) Saturate each mini tress in a solution containing 1% (w/w) active for 30 minutes
d) For a control, saturate a mini tress in DI water for 30 minutes
e) After 30 minutes rinse tress under lukewarm water for 30 seconds
f) Use a hair dryer to dry each mini tress using a low heat, high power setting

Hair tensile test data was measured as described in the Test Method section above taking 50 fibres from each mini tress.

The results were compared to the DI water control. The average results are given in Table 4 below.

TABLE 4
Hair Test Results Rinse-Off Treatment

| Sample | Treatment Composition | Break Stress (gmf/sq micron) |
|---|---|---|
| Control | DI Water | 1.907E−02 |
| 1 | Amino Compound B without | 1.949E−02 |
| | itaconic modification | |
| 2 | 100 mol % Itaconic Modified | *1.966E−02 |
| | Amino Compound A | |

*denotes a significant improvement compared to the control (Student's t test with 95% confidence interval)

The results are consistent between Table 3 & Table 4 despite the tresses being rinsed in Table 4. Amino Compound B shows an improvement compared to the control. Itaconic modified Amino Compound A shows further improvement, significantly different to the control.

### Example 5

Hair samples were treated and tensile tested using the following procedure:
a) Bleach European brown, non-damaged hair to the desired damage level using a commercial bleaching system (Clynol Viton Cream Peroxide 12% and Clynol Viton Blue Powder Bleach)
b) Divide a single hair tress into multiple mini tresses to undergo different treatments and be compared
c) Saturate each mini tress in a solution containing 1% (w/w) active for 30 minutes d) For a control, saturate a mini tress in DI water for 30 minutes
e) Use a hair dryer to dry each mini tress using a low heat, high power setting

Hair tensile test data was measured as described in the Test Method section above taking 50 fibres from each mini tress. Each test was repeated (except Sample 7) with mini tresses from the same original hair tress to give 2×50 fibres for each treatment, total 100 fibres.

The results were compared to the DI water control. The average results are given in Table 5 below.

TABLE 5
Hair Test Results from Different Modification Levels

| Sample | Treatment Composition | Break Stress (gmf/sq micron) |
|---|---|---|
| Control | DI Water | 1.826E−02 |
| 5 | 50 mol % Itaconic Modified | 1.855E−02 |
| | Amino Compound A | |
| 6 | 75 mol % Itaconic Modified | *1.909E−02 |
| | Amino Compound A | |
| 7# | 90 mol % Itaconic Modified | *1.899E−02 |
| | Amino Compound C | |

*denotes a significant improvement compared to the control (Student's t test with 95% confidence interval)
#denotes a test of 50 fibres

Table 5 displays the increase in break stress when hair is treated with itaconic modified hydrolysed potato protein. 75% and 90% itaconic modified hydrolysed potato protein shows a significant improvement compared to the control.

It is to be understood that the invention is not to be limited to the details of the above embodiments, which are described by way of example only. Many variations are possible.

1. A composition comprising the reaction product of:
a) itaconic anhydride; and
b) an amino compound comprising at least one free amino group, wherein the amino compound is selected from the group consisting of amino acids, peptides and hydrolysed proteins;
wherein the composition comprises itaconic modified amino compound, wherein the itaconic modified amino compound is the reaction product of a) and b), and wherein the amount of itaconic anhydride used in the reaction is calculated to react with 10 mol % to 100 mol % of the free amino groups of the amino compound.

2. The composition according to claim 1 further comprising water.

3. The composition according to claim 1 further comprising itaconic acid.

4. The composition according to claim 1 further comprising a preservative.

5. The composition according to claim 1 wherein the amino compound is a hydrolysed protein.

6. The composition according to claim 1 wherein the amino compound is not an individual amino acid.

7. The composition according to claim 1 wherein the amino compound has a weight average molecular weight in the range from 100 Da to 5000 Da.

8. The composition according to claim 1 which does not comprise an organosilane, organosilicon or silicone compound.

9. A The composition according to claim 1 which does not comprise a quaternary ammonium compound.

10. A personal care formulation comprising the composition according to claim 1.

11. A personal care formulation comprising the reaction product of itaconic anhydride and hydrolysed protein, wherein the personal care formulation comprises from 0.1 to 10 wt % of the reaction product of itaconic anhydride and hydrolysed protein, on the basis of the total weight of the formulation, and wherein the personal care formulation is suitable for topical application.

12. A hair care formulation comprising the composition according to claim 1 and a further hair care ingredient.

13. A skin care formulation comprising the composition according to claim 1 and a further skin care ingredient.

14. A method of treating hair or skin, comprising the step of applying the composition according to claim 1 to the hair or skin.

15. The method according to claim 14 wherein the composition provides a benefit to the hair or skin and wherein the benefit is selected from cleaning and conditioning.

16. A method of treating hair, comprising the step of applying the composition according to claim 1 to the hair, wherein the composition provides a benefit to the hair and wherein the benefit is selected from hair strengthening, cleaning, conditioning, detangling, repair, shine, vibrancy, softness, anti-frizz, cuticle smoothing, elasticity, humidity control, curl retention, heat protection, improved movement, volume and styling.

17. Use of a composition according to claim 1 to treat hair or skin to provide a benefit to the hair or skin selected from cleaning and conditioning.

18. Use of a composition according to claim 1 to treat hair to provide a benefit to the hair selected from hair strengthening, cleaning, conditioning, detangling, repair, shine, vibrancy, softness, anti-frizz, cuticle smoothing, elasticity, humidity control, curl retention, heat protection, improved movement, volume and styling.
